(19) United States
(12) Patent Application Publication
(10) Pub. No.: US 2020/0371108 A1
(43) Pub. Date: Nov. 26, 2020

(54) HOMOGENEOUS DETECTION METHOD

(71) Applicant: SHENZHEN LANGJI LIFE SCIENCE AND TECHNOLOGY CO., LTD, Shenzhen (CN)

(72) Inventor: Jianliang ZHANG, Shenzhen (CN)

(21) Appl. No.: 16/768,258

(22) PCT Filed: Nov. 29, 2017
(86) PCT No.: PCT/CN2017/113570

§ 371 Date: May 29, 2020

(51) Int. Cl. G01N 33/58; G01N 21/64

(52) U.S. Cl. CPC G01N 33/581; G01N 2021/6439; G01N 21/6428

(57) ABSTRACT

A homogeneous phase detection method. The homogeneous phase detection method comprises the following steps: providing an aptamer and an enzyme, the aptamer being capable of specifically identifying an analyte, connecting the aptamer to the enzyme to produce an aptamer-enzyme compound; configuring analyte standard solutions of different concentrations, adding the aptamer-enzyme compound and an enzyme action substrate to the analyte standard solutions for enzyme-catalyzed reactions, measuring enzyme-catalyzed reaction signals, acquiring a formula for the enzyme-catalyzed reaction signals and the content of the analyte; adding the aptamer-enzyme compound and the substrate into a sample solution containing the analyte for an enzyme-catalyzed reaction, measuring an enzyme-catalyzed reaction signal, and calculating the content of the analyte in the sample solution on the basis of the formula. The homogeneous phase detection method is characterized by high sensitivity, great repeatability, strong anti-interference properties, fast detection rate, and inexpensiveness, allows the detection of various biological molecules, and is very widely applicable.

### TECHNICAL FIELD

The present disclosure relates to the field of biological detection technology, and particularly relates to a homogeneous detection method.

### BACKGROUND

Currently, detection technologies for biological macromolecules include latex enhanced turbidimetric immunoassay (LETIA), and chemiluminescent immunoassay (CLIA). LETIA is a method in which monoclonal antibodies crosslinked to the surface of high molecular weight latex particles quickly aggregate with antigens (if present) within a short time so as to alter the absorbance of a reaction solution. There is a linear relationship between the change in the absorbance of a reaction solution and the concentration of the antigen to be tested in a certain range, and thus the change in the absorbance of a reaction solution can be used to reflect the concentration of the antigen to be tested. CLIA, which is an immunoassay in which antigens or antibodies are directly labeled with chemiluminescent labels, has a very high sensitivity and good linear range.

Detection technologies for small chemical molecules include cloned enzyme donor immunoassay (CEDIA), enzyme multiplied immunoassay (EMIT), and fluorescence polarization immunoassay (FPIA). In CEDIA, two fragments of β-galactosidase which are prepared by recombination DNA technology, a big fragment (enzyme acceptor, EA) and a small fragment (enzyme donor, ED), show no enzymatic activity alone but have enzymatic activity when combined together under a suitable condition. Cloned enzyme donor immunoassay is a homogeneous enzymatic immunoassay which makes use of the features of the two fragments. After binding to an antibody, a hapten labeled with ED cannot bind to EA for steric hindrance. When a sample contains the hapten to be detected, the hapten to be detected may compete with the hapten labeled with ED for binding the antibody, so as to enable the ED which has been inhibited to be free to bind EA to exert enzymatic activity. CEDIA is mostly used for the detection of drugs and small molecules. It has also been reported in some patent documents that certain epitopes of protein molecules are linked to ED fragment to enable the detection of biological macromolecules. However, currently, the tests of CEDIA are performed through competition method, and the concentration of the substance to be detected is in proportion to the enzymatic activity. The fundamental of EMIT is in that a hapten binds to an enzyme to form a hapten labeled with the enzyme which maintain the activities of the hapten and the enzyme. Upon the binding of the hapten labeled with the enzyme to an antibody, the enzyme contacts closely with the antibody, such that the active center of the enzyme is affected and the activity is inhibited. When a sample contains the hapten to be detected, the hapten to be detected may compete with the hapten labeled with enzyme for binding the antibody to restore the activity of the enzyme which has been inhibited. The tests of EMIT are performed through competition method, and the concentration of the substance to be detected is in proportion to the enzymatic activity. The agents in FPIA are small molecule to be detected labeled with fluorescein and antibodies against drug. The mode in FPIA is homogeneous competition method. The principle of FPIA is as follows: a fluorescent material may absorb light energy to transit into excited state after irradiation of a polarized blue light in a single plane (wavelength 485 nm). Upon restoration to ground state, energy is released and a polarized fluorescence in a single plane is emitted (wavelength 525 nm). The intensity of the polarized fluorescence is inversely proportional to the rotation rate of molecules in the fluorescent material in excited state. Macromolecules have a low rotation rate and thus emit a polarized fluorescence having a high intensity. Small molecules have a high rotation rate and thus emit a polarized fluorescence having a low intensity. Fluorescent molecules are connected to the small molecules to be detected, and antibodies against the small molecules to be detected are added in detection environment. When the small molecules are present in the detection environment, the small molecules may compete with the small molecules labeled with the fluorescent molecules for binding the antibodies, which releases part of the materials to be detected which have been labeled with the fluorescent molecules and lowers the intensity of the polarized light in the detection environment. This method is a competition method, and the concentration of the substance to be detected is inversely proportional to the intensity of polarized light. Based on this phenomenon, FPIA is established for the detection of small molecules especially drugs.

Among the assays mentioned above, LETIA has only an analysis sensitivity up to 0.1 mg/L, and cannot be used for the analyses which require higher sensitivity. CLIA, which is a non-homogeneous assay in which washing and separation steps are included, has a low detection rate, a high detection cost, and a poor repeatability due to being a non-homogeneous assay. Further, due to the use of antibodies, the above-mentioned methods may have interference such as rheumatoid factor (RF) and human anti-animal immunoglobulin (HAAA), which may severely affect the accuracy of detection results. CEDIA, EMIT, and FPIA, which are all competition methods suitable for the detection of small molecules, are very limited for the detection of macromolecules. In addition, under the same conditions, a competition method has a lower sensitivity as compared to that of a non-competition method. Therefore, there is a need to improve the art.

### SUMMARY

The object of the present disclosure is to remedy the defects mentioned above in the prior art, and to provide a homogeneous detection method to solve the technical problems of the existing detection methods of poor sensitivity and repeatability, high cost, and thus limited application.

In order to achieve the object mentioned above, the present disclosure employs technical solutions as follows:

The present disclosure provides a homogeneous detection method, comprising the steps of:

providing an aptamer and an enzyme, the aptamer being capable of specifically recognizing a substance to be detected, and linking the aptamer to the enzyme to form an aptamer-enzyme complex;

preparing standard solutions of the substance to be detected with different concentrations, adding the aptamer-enzyme complex and a substrate of the enzyme into the standard solutions of the substance to be detected to perform an enzymatic reaction, measuring a signal of the enzymatic reaction, and obtaining an equation of the signal of the enzymatic reaction and a content of the substance to be detected; and

adding the aptamer-enzyme complex and the substrate into a sample solution containing the substance to be detected to perform the enzymatic reaction, measuring a signal of the enzymatic reaction, and calculating the content of the substance to be detected in the sample solution according to the equation.

The homogeneous detection method provided by the present disclosure is a brand new homogeneous non-competition method, which labels an enzyme with an aptamer capable of specifically recognizing a substance to be detected. Based on the signal of the enzymatic reaction in the standard solution of the substance to be detected, an equation of the signal of the enzymatic reaction and the content of the substance to be detected is deduced. Then, the signal of the enzymatic reaction in a sample solution is measured, and the content of the substance to be detected in the sample solution is calculated according to the equation. As the signal of the enzymatic reaction is proportional to the content of the substance to be detected, the content of the substance to be detected can be quickly calculated from the signal of the enzymatic reaction in a sample solution. Thus, in comparison with the prior art, the present disclosure has a high sensitivity, good repeatability, strong anti-interference capability, fast detection, low cost, and wide application for the detection of various biological molecules.

### BRIEF DESCRIPTION OF THE DRAWINGS

FIG. 1 shows a schematic diagram of C-reactive protein assay in Example 1 of the present disclosure;

FIG. 2 shows a schematic diagram of C-reactive protein assay in Example 2 of the present disclosure.

### DETAILED DESCRIPTION OF THE EMBODIMENTS

In order to further clarify the technical problem to be solved, technical solutions, and advantages of the present disclosure, hereinafter the present disclosure is further explained in details in combination with the figures and embodiments. It should be appreciated that the specific embodiments described herein is used only to explain the present disclosure, but in no way to limit the scope of the present disclosure.

The embodiments of the present disclosure provide a homogeneous detection method, which includes the following steps of:

S01, providing an aptamer and an enzyme, the aptamer being capable of specifically recognizing a substance to be detected, and linking the aptamer to the enzyme to form an aptamer-enzyme complex;

S02, preparing standard solutions of the substance to be detected with different concentrations, adding the aptamer-enzyme complex and a substrate of the enzyme into the standard solutions of the substance to be detected to perform an enzymatic reaction, measuring a signal of the enzymatic reaction, and obtaining an equation of the signal of the enzymatic reaction and a content of the substance to be detected; and

S03, adding the aptamer-enzyme complex and the substrate into a sample solution containing the substance to be detected to perform the enzymatic reaction, measuring a signal of the enzymatic reaction, and calculating the content of the substance to be detected in the sample solution according to the equation.

The homogeneous detection method provided by the present disclosure is a brand new homogeneous non-competition method, which labels an enzyme with an aptamer capable of specifically recognizing a substance to be detected. Based on the signal of the enzymatic reaction in the standard solution of the substance to be detected, an equation (which, in specific embodiments, may be linear equation or non-linear equation) of the signal of the enzymatic reaction and the content of the substance to be detected is deduced. Then, the signal of the enzymatic reaction in a sample solution is measured, and the content of the substance to be detected in the sample solution is calculated according to the equation. As the signal of the enzymatic reaction is proportional to the content of the substance to be detected, the content of the substance to be detected can be quickly calculated from the signal of the enzymatic reaction in a sample solution.

As the aptamer has a lower molecular weight, when a specific aptamer is linked to an enzyme or enzyme fragments having complementary activity (enzyme donor or enzyme acceptor), the aptamer per se will not affect the activity of the enzyme or the complementary activity of the enzyme fragments. Linking the aptamer to the enzyme will not affect the activity of the enzyme or the complementary activity of the enzyme fragments, either. When a substance to be detected is contained in a sample solution, the substance to be detected binds directly and specifically to the aptamer. Due to the steric effects, the activity of the enzyme or the complementary activity of the enzyme fragments will be lost. The higher the concentration of the substance to be detected, the lower the activity of the enzyme. The concentration of the substance to be detected is inversely proportional to the activity of the enzyme. Therefore, the concentration of the substance to be detected in the sample solution can be quickly calculated after the signal of the enzymatic reaction (corresponding to the activity of the enzyme) is detected. This homogeneous detection method has a high sensitivity, good repeatability, strong anti-interference capability, fast detection, low cost, and wide application for the detection of various biological molecules.

Specifically, in the homogeneous detection method of the embodiments of the present disclosure, the enzyme may be an enzyme having a catalytic activity, i.e., a holoenzyme, or may be enzyme donor and enzyme acceptor having complementary activity (the enzyme donor and the enzyme acceptor form a holoenzyme having a catalytic activity). Further, the enzyme may be a natural enzyme or an artificial enzyme. The artificial enzyme may be one which is obtained by the genetic engineering modification of the holoenzyme or enzyme fragments mentioned above to have a modified activity or specificity to optimize the performance of the present detection method, such as sensitivity, linear range, detection specificity, and stability.

When the enzyme is a holoenzyme, the aptamer is linked to the holoenzyme to obtain the aptamer-enzyme complex in the step S01, and then an enzymatic reaction is performed. Of course, when the enzyme is an enzyme donor and an enzyme acceptor having complementary activity, the aptamer may be individually linked to the enzyme donor or the enzyme acceptor, and then a homogeneous detection is carried out. All these three approaches can achieve the effect of the present disclosure, and thus fall within the scope of the present disclosure. Specifically, the two approaches in which the aptamer is separately linked to the enzyme donor or the enzyme acceptor are as follows:

A homogeneous detection method includes the steps of:

S011: providing an aptamer capable of specifically recognizing a substance to be detected and an enzyme including an enzyme donor and an enzyme acceptor which have complementary activity, and linking the aptamer to the enzyme donor to obtain an aptamer-enzyme donor complex;

S012: preparing standard solutions of the substance to be detected with different concentrations, adding the aptamer-enzyme donor complex, the enzyme acceptor, and a substrate of the enzyme into the standard solutions of the substance to be detected to perform an enzymatic reaction, measuring a signal of the enzymatic reaction, and obtaining an equation of the signal of the enzymatic reaction and a content of the substance to be detected; and

S013: adding the aptamer-enzyme donor complex, the enzyme acceptor, and the substrate into a sample solution containing the substance to be detected to perform the enzymatic reaction, measuring a signal of the enzymatic reaction, and calculating the content of the substance to be detected in the sample solution according to the equation.

A homogeneous detection method includes the steps of:

S021: providing an aptamer capable of specifically recognizing a substance to be detected and an enzyme including an enzyme donor and an enzyme acceptor which have complementary activity, and linking the aptamer to the enzyme acceptor to obtain an aptamer-enzyme acceptor complex;

S022: preparing standard solutions of the substance to be detected with different concentrations, adding the aptamer-enzyme acceptor complex, the enzyme donor and a substrate of the enzyme into the standard solutions of the substance to be detected to perform an enzymatic reaction, measuring a signal of the enzymatic reaction, and obtaining an equation of the signal of the enzymatic reaction and a content of the substance to be detected; and

S023: adding the aptamer-enzyme acceptor complex, the enzyme donor and the substrate into a sample solution containing the substance to be detected to perform the enzymatic reaction, measuring a signal of the enzymatic reaction, and calculating the content of the substance to be detected in the sample solution according to the equation.

Specifically, in step S011, when the aptamer is linked to the enzyme donor, the aptamer-enzyme donor complex in step S012 or S013 binds to the enzyme acceptor to form the aptamer-enzyme complex in S01. In a similar way, in step S021, when the aptamer is linked to the enzyme acceptor, the aptamer-enzyme acceptor complex in step S022 or S023 binds to the enzyme donor to form the aptamer-enzyme complex in S01.

Further, in the above-mentioned homogeneous detection method, the aptamer includes at least one of nucleic acid aptamer, polypeptide aptamer and peptide nucleic acid aptamer. The molecular weight of the aptamer is generally less than 10 kDa. In the embodiments of the present disclosure, the molecular weight of the aptamer is preferably less than 3 kDa, in which range the aptamer may have optimal performance. In one specific embodiment, the aptamer is a polypeptide aptamer having an amino acid sequence as shown by SEQ ID NO.1: EWACNDRGFNCQLQR. More preferably, the aptamer is a modified aptamer. Modification includes at least one of cyclizing modification (e.g., forming an intramolecular disulfide), methylation modification, and phosphorylation modification. The modification of the aptamer may further increase the stability, affinity, and specificity of the aptamer. Of course, if it is the small-molecule antagonists of the substance to be detected or the derivatives thereof, which also have the same function as the aptamer, may also fall within the scope of the aptamer in the embodiments of the present disclosure.

Further, in the above-mentioned homogeneous detection method, the substance to be detected includes at least one of protein, liposome, hormone, nucleic acid, virus, bacteria, fungus, cell and tissue. That is, the homogeneous detection method of the embodiments of the present disclosure may be used to detect various biological macromolecules, cells, or tissues, which generally have a molecular weight greater than 10 kDa. The higher the molecular weight, the higher the detection sensitivity. Preferably, for better detection sensitivity, the substance to be detected has a molecular weight greater than 50 kDa.

Further, in the above-mentioned homogeneous detection method, the enzyme includes, but not limited to, at least one of glucose-6-phosphate dehydrogenase, β-galactosidase, peroxidase, luciferase, alkaline phosphatase and fluorescin (such as green fluorescin and red fluorescin). The substrate of the enzyme is any one of chromogenic substrate, luminescent substrate and fluorescent substrate, which may be suitably determined according to the requirements for sensitivity, linear range, and the like. The signal of the enzymatic reaction may be any one of colorimetric signal, luminescent signal and fluorescent signal. In the embodiments of the present disclosure, the corresponding substrates of the preferable enzymes β-galactosidase and gluco se-6-phosphate dehydrogenase are o-nitrobenzene beta-D-galactopyranoside and glucose-6-phosphate, respectively. Meanwhile, β-galactosidase is divided into an ED fragment and an EA fragment. The ED fragment is co-expressed in fusion with the aptamer. Glucose-6-phosphate dehydrogenase is linked, as a holoenzyme, to the aptamer.

Further, in the above-mentioned homogeneous detection method, the enzyme is linked to the aptamer by any one of chemical coupling, affinity adsorption (like biotin-avidin linkage), and gene fusion expression. The aptamer and the enzyme or the enzyme fragment can be obtained by chemical synthesis or gene expression, preferably by gene fusion expression of the enzyme liked to the aptamer. More preferably, the enzyme may be linked to the aptamer via a linker peptide. That is, a linker peptide can be added between the aptamer and the holoenzyme or the enzyme fragment. The aptamer and the holoenzyme or the enzyme fragment can retain their independent biological features with the linker peptide. In one embodiment of the present disclosure, an ED-CA fusion protein is obtained by fusion expression of an ED fragment of β-galactosidase linked to an aptamer recognizing C-reactive protein via a linker peptide (which has a sequence of: GGGGS).

Further, in the above-mentioned homogeneous detection method, the enzyme is linked to one or more aptamers. That is, the enzyme may be linked to one or more of the aptamers for the same substance to be detected, thus achieving the detection of one substance to be detected. Labeling with one or more of the aptamers for the same substance to be detected has a significant advantage in avoiding false negative. Of course, aptamers for different substances to be detected may also be selected for labeling different enzymes or enzyme fragments, thus achieving simultaneous detection of multiple substances to be detected. All the above-mentioned methods fall within the scope of the present disclosure.

Further, in the above-mentioned homogeneous detection method, during the enzymatic reaction, additional chemical substances are further added, which include, but not limited to, at least one of surfactant, cyclodextrin, bovine serum albumin (BSA), casein, amino acid, chelating agent, nucleotide, hydrophilic macromolecule, reducing agent, oxidizing agent, preservative, buffer salt, polysaccharide, alcohol and metal ion. The addition of these substances may possibly alter the complementary activity of the enzyme fragment with which the aptamer is labeled, or alter the affinity between the enzyme with which the aptamer is labeled and the substance to be detected, to decrease the interference of the analogs of the substance to be detected, or to improve the features such as stability, anti-interference capability of the prepared agents.

Further, in the above-mentioned homogeneous detection method, the homogeneous detection includes any one of tube detection, plate detection, microfluidic detection, and chromatographic detection. Further, a kit of liquids, powders, or the combination of the two may be prepared depending on the agents used in the homogeneous detection method.

Further, in one embodiment of the present disclosure, the two fragments of the enzyme (an enzyme donor fragment and an enzyme acceptor fragment) may be substituted by two different fluorophores (a donor fluorophore and an acceptor fluorophore) which may generate fluorescence resonance energy transfer, respectively, which may also achieve the same technical effect as those of the detection methods mentioned above.

Specifically, the alternative homogeneous detection method includes the following steps of:

E01: providing an aptamer capable of specifically recognizing a substance to be detected, and a donor fluorophore and an acceptor fluorophore that being capable of undergoing a fluorescence resonance energy transfer;

E02: preparing standard solutions of the substance to be detected with different concentrations, linking the donor fluorophore and the acceptor fluorophore to the aptamer, then adding the donor fluorophore and the acceptor fluorophore into the standard solutions of the substance to be detected, measuring a fluorescence intensity of the donor fluorophore, and obtaining an equation of the fluorescence intensity and the content of the substance to be detected; and

E03: adding the donor fluorophore linked to the aptamer and the acceptor fluorophore linked to the aptamer into a sample solution containing the substance to be detected, measuring a fluorescence intensity of the donor fluorophore, and calculating the content of the substance to be detected in the sample solution according to the equation.

Fluorescence resonance energy transfer is a phenomenon in which if there are two different fluorophores, the emission spectrum of one fluorophore (donor) is partly overlapped with the absorption spectrum of the other fluorophore (acceptor), when the distance between the two fluorophores is appropriate (generally less than 100 Å), it can be observed that fluorescent energy is transferred from the donor to the acceptor, i.e., when excitation is performed at the excitation wavelength of the former fluorophore, a fluorescence emitted by the latter fluorophore can be observed. The donor fluorophore and the acceptor fluorophore may be labeled with one or more aptamers, respectively. When a substance to be detected is added, the aptamers on the two fluorophores can form a sandwich structure with the substance to be detected, and shorten the distance between the two fluorophores, and thus enable a fluorescence resonance energy transfer. The fluorescence intensity emitted by the fluorophore is detected from a signal of the fluorescence resonance energy transfer. The fluorescence intensity is proportional to the concentration of the substance to be detected. Thus, the concentration of the substance to be detected may be quickly calculated.

Further, in the homogeneous detection method, the donor fluorophore and the acceptor fluorophore are any of paired fluorescins, paired organic dyes, and paired semiconductor quantum dots. Commercially available fluorescent dyes which can be used as the two paired fluorophores in the present homogeneous detection method include, but not limited to, fluorescins (e.g., paired cyan fluorescin and yellow fluorescin), organic dyes (e.g., Cyanine dyes of GE Healthcare), semiconductor quantum dots, and the like.

In this homogeneous detection method based on fluorescence resonance energy transfer signal, the aptamer may be the same as in the above-mentioned method based on the signal of the enzymatic reaction. The aptamer may include at least one of nucleic acid aptamer, polypeptide aptamer and peptide nucleic acid aptamer, or may be modified. The substance to be detected may be the same as in the above-mentioned method, and may include at least one of protein, liposome, hormone, nucleic acid, virus, bacteria, fungus, cell and tissue. The donor fluorophore and the acceptor fluorophore may also be linked to the aptamer by any one of chemical coupling, affinity adsorption (like biotin-avidin linkage), and gene fusion expression. Of course, for semiconductor quantum dots, it is chemical coupling or affinity adsorption. In summary, other steps are the same except for the requirement for the addition of an enzyme substrate.

The present disclosure has been subjected to a number of tests, a portion of which is described in further detail with reference to the results of the tests, and is described in detail below in conjunction with specific embodiments.

### Example 1

Reagents and steps used in a detection method for C-reactive protein are as follows:

Materials: sodium phosphate (purchased from Sinopharm), EDTA (ethylenediamine tetraacetic acid, purchased from Sinopharm), ONPG (o-nitrophenyl-β-D-galactopyranoside, purchased from Sigma), Tween-20 (purchased from Sinopharm), preservative proclin-300 (purchased from Sigma), C-reactive protein (purchased from Nanjing Leading Biotechnology), ED-CA fusion protein (constructed and expressed by our company, having as sequence as shown in SEQ ID NO.2), EA enzyme fragment (constructed and expressed by our company, deleted the amino acids at positions 11-41 of wild-type β-galactosidase).

Preparation of Reagent 1: 200 mM of sodium phosphate, pH was adjusted to 7.3, 5 mM of EDTA, 0.1% proclin-300, 1% BSA, and 0.05 uM ED-CA fusion protein were added.

Preparation of Reagent 2: 200 mM of sodium phosphate, pH was adjusted to 7.3, 5 mM EDTA, 0.1% proclin-300, 1% BSA, 0.05 uM EA enzyme fragment, and 0.2 g/L ONPG were added.

C-reactive protein standard solution: Four standard solutions having a concentration of 1 mg/L, 10 mg/L, 100 mg/L, 1000 mg/L, respectively, were prepared.

ED-CA fusion protein was obtained by fusion expression (ED-CA) of the ED fragment of β-galactosidase linked to an aptamer recognizing C-reactive protein (SEQ ID NO.1: EWACNDRGFNCQLQR) via a linker peptide (sequence: GGGGS). SEQ ID NO.2 is as follows: ITDSLAVVLQRRDWENPGVTQLNRLAAHPPFASWRNSEEARTDRPS QQLRSLNGGGGGSEWACNDRGFNCQLQR.

Detection step: In a reaction vessel, 2 ul of C-reactive protein standard solution was added, and then 100 ul of Reagent 1 was added, and incubated at 37° C. for 5 mins. Then, 100 ul of Reagent 2 was added to perform an enzymatic reaction at 37° C. Change in absorbance at 415 nm was detected. Rate of change in absorbance (reactivity) is inversely proportional to the concentration of C-reactive protein. A standard curve (i.e., a relational equation) was established with the concentrations of the C-reactive protein standard solutions and the reactivity. The reactivity of a sample solution to be tested was measured, and the concentration of C-reactive protein in the sample solution may be calculated according to the standard curve.

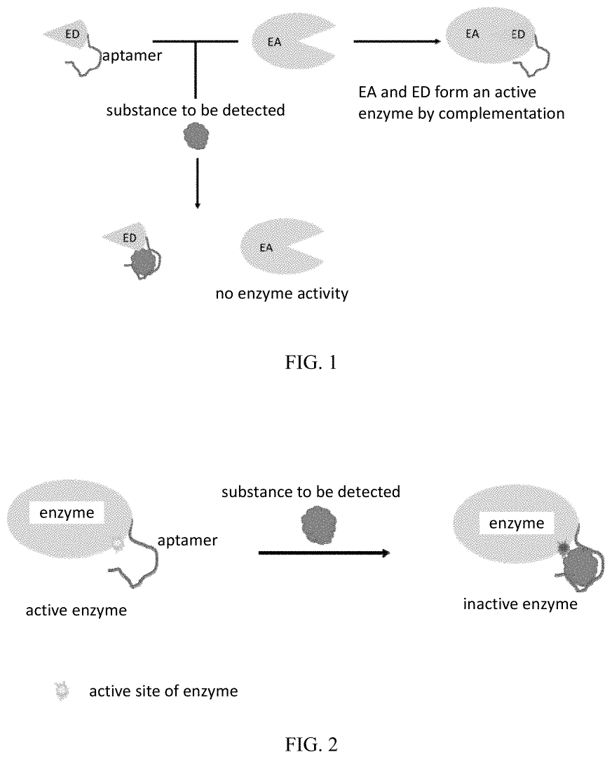

The principle of this Example is shown in FIG. 1: As very few amino acids of the aptamer is linked to the ED fragment, the formation of an active enzyme by the complementation of ED-CA and EA is not affected. When C-reactive protein is present in a detection environment, the C-reactive protein binds to the ED-CA. As C-reactive protein has a molecular weight of about 110 kd, the binding of the two hinders the binding of ED-CA and EA fragment, which can thus not form an active enzyme. The higher the concentration of C-reactive protein, the more inactive enzyme, and the lower the value of the enzymatic reaction signal. The concentration of C-reactive protein may be calculated from the value of the enzymatic reaction signal.

### Example 2

Reagents and steps used in a detection method for C-reactive protein are as follows:

Materials: TRIS (trihydroxymethylaminomethane, purchased from Aladdin), EDTA (purchased from Sinopharm), glucose-6-phosphate dehydrogenase (purchased from Roche), glucose-6-phosphate (purchased from Roche), oxidative coenzyme II (purchased from Roche), preservative proclin-300 (purchased from Sigma), MES (2-morpholinethanesulfonic acid, purchased from Sigma), C-reactive protein (purchased from Nanjing Leading Biotechnology), EDC (1-(3-dimethylaminopropyl)-3-ethylcarbodiimide hydrochloride, purchased from Sigma).

Aptamer labeled with glucose-6-phosphate dehydrogenase (G6PD-CA): 5 g/L IVIES buffer solution was prepared. pH was adjusted to 6.5. 50 mg/L of glucose-6-phosphate dehydrogenase and 2 mg/L of C-reactive protein aptamer (SEQ ID NO.1: EWACNDRGFNCQLQR) were added. After mixing thoroughly, 100 mg of EDC was added and reacted at 25° C. for 1 hour. 10 g/L of BSA was added to stop the reaction. Dialysis using a 20 kd dialysis bag was performed overnight, and unreacted C-reactive protein was removed.

Preparation of Reagent 1: 200 mM of TRIS buffer, pH was adjusted to 8.0, 10% G6PD-CA, 0.1% proclin-300, and 1% BSA were added.

Preparation of Reagent 2: 2 g/L of glucose-6-phosphate, 4 g/L of oxidative coenzyme II, and 0.1% proclin-300 in purified water were added.

C-reactive protein standard solution: Four standard solutions having a concentration of 1 mg/L, 10 mg/L, 100 mg/L, 1000 mg/L, respectively, were prepared.

Detection step: In a reaction vessel, 2 ul of the C-reactive protein standard solution was added, and then 200 ul of Reagent 1 was added, and incubated at 37° C. for 5 mins. Then, 50 ul of Reagent 2 was added to react at 37° C. Change in absorbance at 340 nm was detected. Rate of change in absorbance (reactivity) is inversely proportional to the concentration of C-reactive protein. A standard curve (i.e., a relational equation) was established with the concentrations of the C-reactive protein standard solutions and the reactivity. The reactivity of a sample solution to be tested was measured, and the concentration of C-reactive protein in the sample solution may be calculated according to the standard curve.

The principle of this Example is shown in FIG. 2: The aptamer recognizing C-reactive protein was coupled to glucose-6-phosphate dehydrogenase in a certain ratio via a coupling reagent, such as EDC, to form an enzyme-aptamer complex (G6PD-CA). As the aptamer is very small, it has a very small influence on the catalytic activity of the enzyme. When C-reactive protein is present in a detection environment, C-reactive protein may bind to G6PD-CA. As C-reactive protein has a molecular weight of about 110 kd, it can cover the catalytic center of the enzyme, resulting in a decreased activity of the enzyme. The higher the concentration of C-reactive protein, the lower the activity of the enzyme, and the lower the value of the enzymatic reaction signal. The concentration of C-reactive protein may be calculated from the value of the enzymatic reaction signal.

The foregoing is only a preferred embodiment of the present disclosure and is not intended to limit the present disclosure and any modifications, equivalents, improvements, etc. made within the spirit and principles of the present disclosure shall be covered by the present disclosure.

1. A homogeneous detection method, comprising the following steps of:
providing an aptamer and an enzyme, the aptamer being capable of specifically recognizing a substance to be detected, and linking the aptamer to the enzyme to form an aptamer-enzyme complex;
preparing standard solutions of the substance to be detected with different concentrations, adding the aptamer-enzyme complex and a substrate of the enzyme into the standard solutions of the substance to be detected to perform an enzymatic reaction, measuring a signal of the enzymatic reaction, and obtaining an equation of the signal of the enzymatic reaction and a content of the substance to be detected; and
adding the aptamer-enzyme complex and the substrate into a sample solution containing the substance to be detected to perform the enzymatic reaction, measuring a signal of the enzymatic reaction, and calculating the content of the substance to be detected in the sample solution according to the equation.

2. The homogeneous detection method of claim 1, wherein the enzyme is a holoenzyme, and the aptamer is linked to the holoenzyme to form the aptamer-enzyme complex.

3. The homogeneous detection method of claim 1, wherein the enzyme comprises an enzyme donor and an enzyme acceptor which have complementary activity, the aptamer is linked to the enzyme donor to form an aptamer-enzyme donor complex; and the method comprises:
preparing standard solutions of the substance to be detected with different concentrations, adding the aptamer-enzyme donor complex, the enzyme acceptor, and a substrate of the enzyme into the standard solutions of the substance to be detected to perform an enzymatic reaction, measuring a signal of the enzymatic reaction, and obtaining an equation of the signal of the enzymatic reaction and a content of the substance to be detected; and
adding the aptamer-enzyme donor complex, the enzyme acceptor, and the substrate into a sample solution containing the substance to be detected to perform the enzymatic reaction, measuring a signal of the enzymatic reaction, and calculating the content of the substance to be detected in the sample solution according to the equation.

4. The homogeneous detection method of claim 1, wherein the enzyme comprises an enzyme donor and an enzyme acceptor which have complementary activity, the aptamer is linked to the enzyme acceptor to form an aptamer-enzyme acceptor complex; and the method comprises:
preparing standard solutions of the substance to be detected with different concentrations, adding the aptamer-enzyme acceptor complex, the enzyme donor and a substrate of the enzyme into the standard solutions of the substance to be detected to perform an enzymatic reaction, measuring a signal of the enzymatic reaction, and obtaining an equation of the signal of the enzymatic reaction and a content of the substance to be detected; and
adding the aptamer-enzyme acceptor complex, the enzyme donor and the substrate into a sample solution containing the substance to be detected to perform the enzymatic reaction, measuring a signal of the enzymatic reaction, and calculating the content of the substance to be detected in the sample solution according to the equation.

5. The homogeneous detection method of claim 1, wherein the aptamer comprises at least one selected from the group consisting of nucleic acid aptamer, polypeptide aptamer, and peptide nucleic acid aptamer.

6. The homogeneous detection method of claim 1, wherein the aptamer is a modified aptamer, the modification comprises at least one selected from the group consisting of cyclizing modification, methylation modification, and phosphorylation modification.

7. The homogeneous detection method of claim 1, wherein the aptamer has a molecular weight of <10 kDa; and/or the substance to be detected has a molecular weight of >10 kDa.

8. The homogeneous detection method of claim 1, wherein the substance to be detected comprises at least one selected from the group consisting of protein, liposome, hormone, nucleic acid, virus, bacteria, fungus, cell, and tissue.

9. The homogeneous detection method of claim 1, wherein the enzyme comprises at least one selected from the group consisting of glucose-6-phosphate dehydrogenase, β-galactosidase, peroxidase, luciferase, alkaline phosphatase and fluorescin; and/or
the enzyme comprises natural enzymes or artificial enzymes; and/or
the substrate is any one selected from the group consisting of chromogenic substrate, luminescent substrate, and fluorescent substrate.

10. The homogeneous detection method of claim 1, wherein the enzyme is linked to the aptamer by any one selected from the group consisting of chemical coupling, affinity adsorption, and gene fusion expression.

11. The homogeneous detection method of claim 1, wherein
the enzyme is linked to one or more of the aptamers; and/or
the enzyme is linked to the aptamer via a linker peptide.

12. The homogeneous detection method of claim 1, wherein, during the enzymatic reaction, a chemical substance is further added, and the chemical substance comprises at least one selected from the group consisting of surfactant, cyclodextrin, bovine serum albumin, casein, amino acid, chelating agent, nucleotide, hydrophilic macromolecule, reducing agent, oxidizing agent, preservative, buffer salt, polysaccharide, alcohol, and metal ion.

13. The homogeneous detection method of claim 1, wherein the signal of the enzymatic reaction is any one selected from the group consisting of colorimetric signal, luminescent signal, and fluorescent signal.

14. The homogeneous detection method of claim 1, wherein the homogeneous detection comprises any one selected from the group consisting of tube detection, plate detection, microfluidic detection, and chromatographic detection.

15. (canceled)


SEQUENCE LISTING (3 sequences)

SEQ ID NO: 1 - protein, 15 aa, Artificial Sequence
  Polypeptide adaptamer
EWACNDRGFNCQLQR

SEQ ID NO: 2 - protein, 74 aa, Artificial Sequence
  Synthetic ED-CA fusion protein
ITDSLAVVLQRRDWENPGVTQLNRLAAHPPFASWRNSEEARTDRPSQQLRSLNGGGGGSEWACNDRGFNCQLQR

SEQ ID NO: 3 - protein, 5 aa, Artificial Sequence
  linker peptide
GGGGS